SEQUENCE LISTING (22 sequences)

SEQ ID NO: 1 - DNA, 864 bp, Homo sapiens
  NET-4 oligonucleotide used in cell proliferation assay on SW620 cells
ctttgcccgaagcccggggacgaaccgacggaccgaccgcctggcgcacggacgcgggcgctcgctttgtgttcggggctagcgtcggcgaggcttgagcttgcagcgcgcggcttccctgctttctcgcggccaccccggctccggcggcctcggcgcgcgaggggctggaggtgcgggagccgctctccgccggtcggtccccgcgcggctgagcccaggccgccagcgccgcggccccgtgcggtgtccctgagctcctgctccccgccgggctgctccgagcaacggtgcttcggagctccaaactcgggctgccggggcaagtgtcttcatgaacccagaggatgtccgggaagcactacaagggtcctgaagtcagttgttgcatcaaatacttcatatttggcttcaatgtcatattttggtttttgggaataacatttcttggaattggactgtgggcatggaatgaaaaaggagttctgtccaacatctcttccatcaccgatctcggcggctttgacccagtttggctcttccttgtggtgggaggagtgatgttcattttgggatttgcagggtgcattggagcgctacgggaaaacactttccttctcaagtttttttctgtgttcctgggaattattttcttcctggagctcactgccggagttctagcatttgttttcaaagactggatcaaagaccagctgtatttctttataaacaacaacatcagagcatatcgggatgacattgatttgcaaaacctcatagacttcacccaggaatattggcagtgctgtggggcttttggagctgatgattggaacctaaatatttacttcaattgcacagatt

SEQ ID NO: 2 - DNA, 23 bp, Artificial Sequence
  NET-4 oligonucleotide used in cell proliferation assay on SW620 cells
ctgggtcaaagccgccgagatcg

SEQ ID NO: 3 - DNA, 25 bp, Artificial Sequence
  NET-4 oligonucleotide used in cell proliferation assay on SW620 cells
ttcaggacccttgtagtgcttcccg

SEQ ID NO: 4 - DNA, 25 bp, Artificial Sequence
  NET-4 oligonucleotide used in cell proliferation assay on SW620 cells
ccatgcccacagtccaattccaaga

SEQ ID NO: 5 - DNA, 25 bp, Artificial Sequence
  NET-4 oligonucleotide used in cell proliferation assay on SW620 cells
gctggtcaacttctggtttttgcct

SEQ ID NO: 6 - DNA, 24 bp, Artificial Sequence
  NET-4 oligonucleotide used in cell proliferation assay on SW620 cells
aaactggggcacacagcctttcgt

SEQ ID NO: 7 - DNA, 23 bp, Artificial Sequence
  NET-4 oligonucleotide used in cell proliferation assay on SW620 cells
gctagagccgccgaaactgggtc

SEQ ID NO: 8 - DNA, 25 bp, Artificial Sequence
  NET-4 oligonucleotide used in cell proliferation assay on SW620 cells
gcccttcgtgatgttcccaggactt

SEQ ID NO: 9 - DNA, 25 bp, Artificial Sequence
  NET-4 oligonucleotide used in cell proliferation assay on SW620 cells
agaaccttaacctgacacccgtacc

SEQ ID NO: 10 - DNA, 25 bp, Artificial Sequence
  NET-4 oligonucleotide used in cell proliferation assay on SW620 cells
tccgtttttggtcttcaactggtcg

SEQ ID NO: 11 - DNA, 24 bp, Artificial Sequence
  NET-4 oligonucleotide used in cell proliferation assay on SW620 cells
tgctttccgacacacggggtcaaa

SEQ ID NO: 12 - DNA, 25 bp, Artificial Sequence
  PCR primer
gatcccgcagaagatgtcatcaaca

SEQ ID NO: 13 - DNA, 24 bp, Artificial Sequence
  PCR primer
tctgcagcaatgcaatgcctatga

SEQ ID NO: 14 - DNA, 24 bp, Artificial Sequence
  PCR primer
cgggaaatcgtgcgtgacattaag

SEQ ID NO: 15 - DNA, 24 bp, Artificial Sequence
  PCR primer
tgatctccttctgcatcctgtcgg

SEQ ID NO: 16 - DNA, 25 bp, Artificial Sequence
  Antisense oligonucleotide kRAS 2576AS
gcatgtggaaggtagggaggcaaga

SEQ ID NO: 17 - DNA, 25 bp, Artificial Sequence
  Antisense oligonucleotide kRAS 2576RS
agaacggagggatggaaggtgtacg

SEQ ID NO: 18 - DNA, 12 bp, Artificial Sequence
  Oligonucleotide NET-4 oligo 864 used for in-situ hybridization
cccttgtagtgc

SEQ ID NO: 19 - DNA, 15 bp, Artificial Sequence
  Oligonucleotide NET-4 oligo 865 used for in-situ hybridization
tgaagtatttgatgc

SEQ ID NO: 20 - DNA, 13 bp, Artificial Sequence
  Oligonucleotide NET-4 oligo 866 used for in-situ hybridization
ccgatatgctctg

SEQ ID NO: 21 - DNA, 14 bp, Artificial Sequence
  Oligonucleotide NET-4 oligo 867 used for in-situ hybridization
ttaggttccaatca

SEQ ID NO: 22 - DNA, 14 bp, Artificial Sequence
  Oligonucleotide NET-4 oligo 868 used for in-situ hybridization
cgactttcatactg

(19) United States
(12) Patent Application Publication
(10) Pub. No.: US 2002/0156035 A1
(43) Pub. Date: Oct. 24, 2002

(54) Compositions and methods for treating neoplastic disease using net-4 modulators

(76) Inventors: Christoph Reinhard, Alameda, CA (US); Anne B. Jefferson, Oakland, CA (US); Jill A. Winter, Richmond, CA (US); Filippo Randazzo, Oakland, CA (US)

Correspondence Address: Chiron Corporation, Intellectual Property, P.O. Box 8097, Emeryville, CA, 94662-8097, US

(21) Appl. No.: 09/875,440

(22) Filed: Jun. 5, 2001

Related U.S. Application Data
(60) Provisional application No. 60/209,865, filed on Jun. 7, 2000.

(51) Int. Cl. A61K 48/00; A61K 31/00; C07H 21/02; C07H 21/04; A61K 39/395; C07K 16/46

(52) U.S. Cl. 514/44; 536/23.1; 424/142.1; 530/387.3; 514/1

(57) ABSTRACT

Modulators of NET-4 are provided that reduce the expression or biological activities of NET-4 or the expression of NET-4 mRNA in a mammalian cell. NET-4 modulators include antisense molecules, ribozymes, antibodies and antibody fragments, proteins and polypeptides as well as small molecules. NET-4 modulators find use in compositions and methods for decreasing NET-4 gene expression as well as methods for inhibiting the proliferation of mammalian cells, including tumor cells, and methods for treating neoplastic diseases.

### CROSS-REFERENCE TO RELATED APPLICATIONS

This application claims priority from United States Provisional Patent Application No. 60/209,865 filed Jun. 7, 2000, which is incorporated by reference herein in its entirety.

### TECHNICAL FIELD

The invention provides compositions and methods for inhibiting NET-4 gene expression and/or biological activity. Such compositions and methods find utility in the treatment of neoplastic disease.

### BACKGROUND OF THE INVENTION

NET-4 is a member of the tetraspan transmembrane receptor family. There have been no published reports disclosing the function of the molecule. The tetraspan family is discussed in Maecker, H. T. et al., FASEB J. 11:428-442, 1997. Expression of tetraspan genes in lymphoma cell lines is discussed in Ferrer, M. et al., Clin. Exp. Immunol. 113:346-352, 1998.

Thus, there is a need in the art for correlation between this protein family and essential or pathological processes. The present invention helps to fulfill this need by disclosing the use of NET-4 antisense oligonucleotides to inhibit proliferation of tumor cells.

### SUMMARY OF THE INVENTION

The present invention provides, in one embodiment, inhibitors of NET-4. Inventive inhibitors include, but are not limited to, antisense molecules, ribozymes, antibodies or antibody fragments, proteins or polypeptides as well as small molecules. Exemplary antisense molecules comprise at least 10, 15 or 20 consecutive nucleotides of or hybridize under stringent conditions to the nucleic acid of SEQ ID NO:1. More preferred are antisense molecules that comprise at least 15, 20 or 25 consecutive nucleotides of or hybridize under stringent conditions to the sequence of SEQ ID NO:1. Representative antisense molecules are provided herein as SEQ ID NO:2-6.

In further embodiments, compositions are provided that comprise one or more NET-4 inhibitor in a pharmaceutically acceptable carrier.

Additional embodiments provide methods of decreasing NET-4 gene expression or biological activity.

Each of the methods of the present invention has in common the administration of one or more inventive NET-4 inhibitor to a mammalian cell.

The invention further provides a kit for detecting a colon tumor cell, comprising at least one oligonucleotide selected from the group consisting of SEQ ID NO:18, 19, 20, 21 and 22.

### BRIEF DESCRIPTION OF THE FIGURES

FIG. 1 illustrates the schematic structure of tetraspan proteins. Amino (N) and carboxyl (C) termini and extracellular and transmembrane domains are indicated in boldface. Highly conserved amino acids found in 12 of 18 tetraspan genes, are shown in circles. Highly conserved amino acids found in 14 or more tetraspans are shown in boldface circles. The conserved PXSC motif is located at different positions within EC2 in the various tetraspans, and is therefore indicated with floating arrows. Asterisks indicate conserved charged amino acids within the transmembrane domains.

FIG. 2 shows in situ hybridization analyses of tumor colon tissue (FIG. 2A), tumor lung tissue (FIG. 2B), normal colon tissue (FIG. 2C), and normal lung tissues (FIG. 2D) using a mixture of oligonucleotides consisting of Net-4 864: CCCTTGTAGTGC (SEQ ID NO:18); Net-4 865: TGAAGTATTTGATGC (SEQ ID NO:19); Net-4 866: CCGATATGCTCTG (SEQ ID NO:20); Net-4 867: TTAGGTTCCAATCA (SEQ ID NO:21); and Net-4 868: CGACTTTCATACTG (SEQ ID NO:22).

### DETAILED DESCRIPTION OF THE INVENTION

NET-4 is a member of the tetraspan superfamily of proteins. The proteins are characterized by four transmembrane domains and two extracellular regions. Within the superfamily is a specific family referred to as NET proteins, for “new EST tetraspan” (Serru, V. et al., Biochem. Biophys. Acta 1478:159-163, 2000). Serru et al. reported the existence of seven NET proteins (designated NET-1 through NET-7), and studied expression in a panel of cell lines. NET-4 was expressed in T lymphoid cell lines, but not by most B lymphoid cell lines studied.

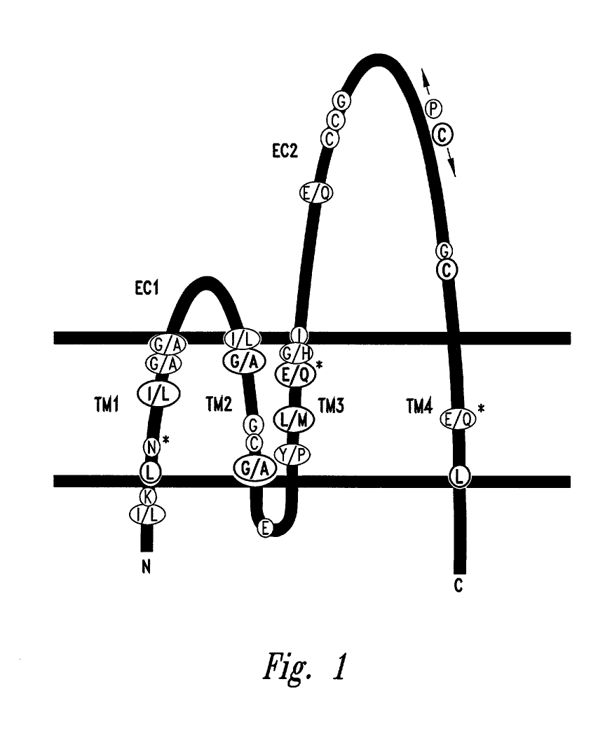

To date there is no published information on a putative function for any of the NET proteins. The superfamily itself was discovered in 1990, and as of 1997, twenty members had been identified. It has been suggested that one of the molecule's functions is to group specific cell-surface proteins, thereby increasing the formation and stability of functional signaling complexes. Maecker, H. T. et al., FASEB J. 11:428-442, 1997. FIG. 1 illustrates the schematic structure of tetraspan proteins. The information available to date indicates that some tetraspan proteins may play an inhibitory role in cancer development or growth, while other proteins are expressed at a higher level in cancer cells. For example, expression of CD9 suppresses motility and metastasis in carcinoma cells (Ikeyama, S. et al., J. Exp. Med. 177:1231-1237, 1993), and CD9 expression is inversely correlated with metastasis in melanoma cells (Si, Z. et al., Int. J Cancer 54:37-43, 1993). Reduction of CD9 expression correlates with poor prognosis in breast carcinoma (Miyake, M. et al., Cancer Res. 56:1244-1249, 1996). Expression of CD82 may suppress metastasis in prostate cancer cell lines (Dong, J. et al., Science 268:884-886, 1995).

The present invention substantially adds to the information about the role of tetraspan proteins in cancer and also provides methods and compositions for modulating cancer cell growth by inhibiting NET-4 expression. According to the invention, NET-4 is overexpressed in 83% of colon cancer patient samples tested using DNA microarray screening. To further investigate the role of NET-4 expression in tumor cell growth, cells of colon cancer cell line SW620 were exposed to a variety of antisense molecules based on the sequence of NET-4. As shown in Tables 2-5 and 7, transfection of SW620 cells with antisense molecules of SEQ ID NO:2-6, respectively, significantly decreased the proliferation of SW620 cells beginning at day 1 and continuing through day 4. Likewise, transfection of MRC9 cells with antisense molecules of SEQ ID NO:5 also significantly decreased the proliferation of MRC9 cells (Table 6).

To determine whether the effect on proliferation was due to a specific effect on NET-4 expression, NET-4 message levels were measured in SW620 cells lines exposed to five different antisense molecules. As shown in Table 1, cells exposed to the antisense oligonucleotides had significantly lower levels of NET-4 mRNA when compared to cells treated with the corresponding reverse control oligonucleotides.

Antisense oligonucleotides based on the polynucleotide sequence of NET-4 therefore are specific inhibitors of NET-4 expression, and this correlates with decreased proliferation of colon cancer cells. Antisense oligonucleotides are suitable for in vivo treatment of colon cancer and other cancers in which increased NET-4 expression plays a role in cancer cell growth, migration, metastasis, and survival. However, the invention is not limited to use of antisense inhibitors. Based on the results herein, other compositions and methods for inhibiting NET-4 expression or for modulating or inhibiting NET-4 function are also suitable for regulating cell proliferation. Because NET-4 is a transmembrane protein, antibodies are particularly suitable for inhibiting its effect.

The present invention is directed generally to modulating NET-4 expression and function, particularly in cancer cells, more particularly in colon cancer cells. More specifically, the invention provides agonists and antagonists of NET-4, including antisense polynucleotides and ribozymes, proteins or polypeptides, antibodies or fragments thereof and small molecules; compositions comprising NET-4 modulators; methods of supplementing chemotherapeutic and/or radiation treatment of a mammalian cell, as well as methods of treating neoplastic disease. Each of these methods has in common the administration to a mammalian cell of one or more NET-4 modulator.

Inhibitors of NET-4 are Effective in Reducing NET-4 Gene Expression

Inventive NET-4 inhibitors include antisense molecules and ribozymes, proteins or polypeptides, antibodies or fragments thereof as well as small molecules. These NET-4 inhibitors share the common feature that they reduce the expression and/or biological activity of NET-4 and, as a consequence, modulate, inhibit, or prevent the growth of cancer cells. In addition to the exemplary NET-4 inhibitors disclosed herein, alternative inhibitors may be obtained through routine experimentation utilizing methodology either specifically disclosed herein or as otherwise readily available to and within the expertise of the skilled artisan.

Antisense Molecules and Ribozymes

NET-4 inhibitors of the present invention include antisense molecules that, when administered to mammalian cells, are effective in reducing, for example, intracellular levels of NET-4 mRNA. Antisense molecules bind in a sequence-specific manner to nucleic acids, such as mRNA or DNA. When bound to mRNA that has complementary sequences, antisense molecules prevent translation of the mRNA (U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. Nucl. Acids Res. 21:3405-3411 (1993), which describes dumbbell antisense oligonucleotides).

Antisense technology can be used to control gene expression through triple-helix formation, which promotes the ability of the double helix to open sufficiently for the binding of polymerases, transcription factors or regulatory molecules. Gee et al., In Huber and Carr, “Molecular and Immunologic Approaches,” Futura Publishing Co. (Mt. Kisco, N.Y.; 1994). Alternatively, an antisense molecule may be designed to hybridize with a control region of the NET-4 gene, e.g., promoter, enhancer or transcription initiation site, and block transcription of the gene; or block translation by inhibiting binding of a transcript to ribosomes. Hirashima et al. in Molecular Biology of RNA: New Perspectives (M. Inouye and B. S. Dudock, eds., 1987 Academic Press, San Diego, p. 401); Oligonucleotides: Antisense Inhibitors of Gene Expression (J. S. Cohen, ed., 1989 MacMillan Press, London); Stein and Cheng, Science 261:1004-1012 (1993); WO 95/10607; U.S. Pat. No. 5,359,051; WO 92/06693; and EP-A2-612844, each of which is incorporated herein by reference.

Briefly, such molecules are constructed such that they are complementary to, and able to form Watson-Crick base pairs with, a region of transcribed NET-4 mRNA sequence. The resultant double-stranded nucleic acid interferes with subsequent processing of the mRNA, thereby preventing protein synthesis.

In general, a portion of a sequence complementary to the NET-4 coding region may be used to modulate gene expression. The sequence of NET-4 cDNA is presented herein as SEQ ID NO:1. Alternatively, cDNA constructs that can be transcribed into antisense RNA may be introduced into cells or tissues to facilitate the production of antisense RNA. Thus, as used herein, the phrase “antisense molecules” broadly encompasses antisense oligonucleotides whether synthesized as DNA or RNA molecules as well as all plasmid constructs that, when introduced into a mammalian cell, promote the production of antisense RNA molecules. An antisense molecule may be used, as described herein, to inhibit expression of mRNA or protein, as well as any other gene that requires NET-4 for its expression.

The present invention relates to antisense oligonucleotides designed to interfere with the normal function of NET-4 polynucleotides. Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases as disclosed in U.S. Pat. No. 5,958,773 and patents disclosed therein. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art a certain degree of routine experimentation may be required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. According to the invention, this experimentation can be performed routinely by transfecting cells with an antisense oligonucleotide using methods described in Example 1. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.) According to the present invention, cultures of SW620 cells were transfected with five different antisense oligonucleotides designed to target NET-4. These oligonucleotides are shown in SEQ ID NO:2-6. The levels of mRNA corresponding to NET-4 were measured in treated and control cells. SEQ ID NO:2-6 caused dramatic decreases in NET-4 mRNA when normalized to actin mRNA levels.

Antisense molecules for use as described herein can be synthesized by any method known to those of skill in this art including chemical synthesis by, for example, solid phase phosphoramidite chemical synthesis. WO 93/01286; U.S. Pat. No. 6,043,090; U.S. Pat. No. 5,218,088; U.S. Pat. No. 5,175,269; and U.S. Pat. No. 5,109,124, each of which is incorporated herein by reference. Alternatively, RNA molecules may be generated by in vitro or in vivo transcription of DNA sequences encoding the NET-4 cDNA, or a portion thereof, provided that the DNA is incorporated into a vector downstream of a suitable RNA polymerase promoter (such as, e.g., T3, T7 or SP6). Large amounts of antisense RNA may be produced by incubating labeled nucleotides with a linearized NET-4 cDNA fragment downstream of such a promoter in the presence of the appropriate RNA polymerase. Such antisense molecules are preferably at least 10, 15 or 20 nucleotides in length. More preferably, antisense molecules are at least 25 nucleotides in length. Within certain embodiments, an antisense molecule of the present invention will comprise a sequence that is unique to the NET-4 cDNA sequence of SEQ ID NO:1 or that can hybridize to the cDNA of SEQ ID NO:1 under conditions of high stringency. Within the context of the present invention, high stringency means standard hybridization conditions such as, e.g., 5×SSPE, 0.5% SDS at 65° C. or the equivalent thereof. See Sambrook et al., supra and Molecular Biotechnology: Principles and Applications of Recombinant DNA, supra incorporated herein by reference.

Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. 17(12):4863-4871 (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein, in: Oligodeoxynucleotides. Antisense Inhibitors of Gene Expression, Cohen, Ed, Macmillan Press, London, pp. 97-117 (1989); Jager et al., Biochemistry 27:7237-7246 (1988)). Possible additional or alternative modifications include, but are not limited to, the addition of flanking sequences at the 5′ and/or 3′ ends and/or the inclusion of nontraditional bases such as inosine, queosine and wybutosine, as well as acetyl- methyl-, thio- and other modified forms of adenine, cytidine, guanine, thymine and uridine.

Within alternate embodiments of the present invention, NET-4 inhibitors may be ribozymes. A ribozyme is an RNA molecule that specifically cleaves RNA substrates, such as mRNA, resulting in specific inhibition or interference with cellular gene expression. As used herein, the term “ribozymes” includes RNA molecules that contain antisense sequences for specific recognition, and an RNA-cleaving enzymatic activity. The catalytic strand cleaves a specific site in a target RNA at greater than stoichiometric concentration.

A wide variety of ribozymes may be utilized within the context of the present invention, including for example, the hammerhead ribozyme (for example, as described by Forster and Symons, Cell 48:211-220 (1987); Haseloff and Gerlach, Nature 328:596-600 (1988); Walbot and Bruening, Nature 334:196 (1988); Haseloff and Gerlach, Nature 334:585 (1988)); the hairpin ribozyme (for example, as described by Haseloff et al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such NET-4 mRNA-specific ribozyme, or a nucleic acid encoding such a ribozyme, may be delivered to a host cell to effect inhibition of NET-4 gene expression. Ribozymes and the like may therefore be delivered to the host cells by DNA encoding the ribozyme linked to a eukaryotic promoter, such as a eukaryotic viral promoter, such that upon introduction into the nucleus, the ribozyme will be directly transcribed.

Proteins and Polypeptides

In addition to the antisense molecules and ribozymes disclosed herein, NET-4 modulators of the present invention also include proteins or polypeptides that are effective in either reducing NET-4 gene expression or in decreasing one or more of NET-4's biological activities. A variety of methods are readily available in the art by which the skilled artisan may, through routine experimentation, rapidly identify such NET-4 inhibitors. The present invention is not limited by the following exemplary methodologies.

Inhibitors of NET-4's biological activities encompass those proteins and/or polypeptides that interfere with cell proliferation, particularly tumor cell proliferation, especially colon cell proliferation. Such interference may occur indirectly through non- or un-competitive inhibition such as via binding to an allosteric site, or by binding to a region that normally binds to another protein. Accordingly, available methods for identifying proteins and/or polypeptides that bind to NET-4 may be employed to identify lead compounds that may, through the methodology disclosed herein, be characterized for their NET-4 inhibitory activity.

A vast body of literature is available to the skilled artisan that describes methods for detecting and analyzing protein-protein interactions. Phizicky, E. M. et al., Microbiological Reviews 59:94-123 (1995) incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive NET-4 inhibitors may be identified through biological screening assays that rely on the direct interaction between the NET-4 protein and a panel or library of potential inhibitor proteins. Biological screening methodologies, including the various “n-hybrid technologies,” are described in, for example, Vidal, M. et al., Nucl. Acids Res. 27(4):919-929 (1999); Frederickson, R. M., Curr. Opin. Biotechnol. 9(1):90-6 (1998); Brachmann, R. K. et al., Curr. Opin. Biotechnol. 8(5):561-568 (1997); and White, M. A., Proc. Natl. Acad. Sci. U.S.A. 93:10001-10003 (1996) each of which is incorporated herein by reference.

The two-hybrid screening methodology may be employed to search new or existing target cDNA libraries for NET-4 binding proteins that have inhibitory properties. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed covalently from the transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest. The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien, C.-T. et al., Proc. Natl. Acad. Sci. U.S.A. 88:9578-9582 (1991); Dalton, S. et al., Cell 68:597-612 (1992); Durfee, T. K. et al., Genes Dev. 7:555-569 (1993); Vojtek, A. B. et al., Cell 74:205-214 (1993); and Zervos, A. S. et al., Cell 72:223-232 (1993). Commonly used reporter genes include the E. coli lacZ gene as well as selectable yeast genes such as HIS3 and LEU2. Fields, S. et al., Nature (London) 340:245-246 (1989); Durfee, T. K., supra; and Zervos, A. S., supra. A wide variety of activation domain libraries are readily available in the art such that the screening for interacting proteins may be performed through routine experimentation.

Suitable bait proteins for the identification of NET-4 interacting proteins may be designed based on the NET-4 cDNA sequence presented herein as SEQ ID NO:1. Such bait proteins include either the full-length NET-4 protein or fragments thereof.

Plasmid vectors, such as, e.g., pBTM116 and pAS2-1, for preparing NET-4 bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

NET-4 modulators of the present invention may alternatively be identified through one of the physical or biochemical methods available in the art for detecting protein-protein interactions.

NET-4 is believed to interact with the other cell surface proteins. Through the protein affinity chromatography methodology, lead compounds to be tested as potential NET-4 inhibitors may be identified by virtue of their specific retention to NET-4 when either covalently or non-covalently coupled to a solid matrix such as, e.g., Sepharose beads. The preparation of protein affinity columns is described in, for example, Beeckmans, S. et al., Eur. J. Biochem. 117:527-535 (1981) and Formosa, T. et al., Methods Enzymol. 208:24-45 (1991). Cell lysates containing the full complement of cellular proteins may be passed through the NET-4 affinity column. Proteins having a high affinity for NET-4 will be specifically retained under low-salt conditions while the majority of cellular proteins will pass through the column. Such high affinity proteins may be eluted from the immobilized NET-4 under conditions of high-salt, with chaotropic solvents or with sodium dodecyl sulfate (SDS). In some embodiments, it may be preferred to radiolabel the cells prior to preparing the lysate as an aid in identifying the NET-4 specific binding proteins. Methods for radiolabeling mammalian cells are well known in the art and are provided, e.g., in Sopta, M. et al., J. Biol. Chem. 260:10353-10360 (1985).

Suitable NET-4 proteins for affinity chromatography may be fused to a protein or polypeptide to permit rapid purification on an appropriate affinity resin. For example, the NET-4 cDNA may be fused to the coding region for glutathione S-transferase (GST) which facilitates the adsorption of fusion proteins to glutathione-agarose columns. Smith et al., Gene 67:31-40 (1988). Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One exemplary tag suitable for the preparation of NET-4 fusion proteins that is presented herein is the epitope for the influenza virus hemaglutinin (HA) against which monoclonal antibodies are readily available and from which antibodies an affinity column may be prepared.

Proteins that are specifically retained on a NET-4 affinity column may be identified after subjecting to SDS polyacrylamide gel electrophoresis (SDS-PAGE). Thus, where cells are radiolabeled prior to the preparation of cell lysates and passage through the NET-4 affinity column, proteins having high affinity for NET-4 may be detected by autoradiography. The identity of NET-4 specific binding proteins may be determined by protein sequencing techniques that are readily available to the skilled artisan, such as Mathews, C. K. et al., Biochemistry, The Benjamin/Cummings Publishing Company, Inc. pp.166-170 (1990).

Antibodies or Antibody Fragments

NET-4 modulators (antagonists and agonists) of the present invention include antibodies and/or antibody fragments that are effective in modulating NET-4 gene expression and/or biological activity. Suitable antibodies may be monoclonal, polyclonal or humanized monoclonal antibodies. Antibodies may be derived by conventional hybridoma based methodology, from antisera isolated from NET-4 inoculated animals or through recombinant DNA technology. Alternatively, inventive antibodies or antibody fragments may be identified in vitro by use of one or more of the readily available phage display libraries. Exemplary methods are disclosed herein.

In one embodiment of the present invention, NET-4 modulators are monoclonal antibodies that may be produced as follows. NET-4 protein may be produced, for example, by expression of NET-4 cDNA in a baculovirus based system. By this method, NET-4 cDNA or a fragment thereof is ligated into a suitable plasmid vector that is subsequently used to transfect Sf9 cells to facilitate protein production. In addition, it may be advantageous to incorporate an epitope tag or other moiety to facilitate affinity purification of the NET-4 protein. Clones of Sf9 cells expressing NET-4 are identified, e.g., by enzyme linked immunosorbant assay (ELISA), lysates are prepared and the NET-4 protein purified by affinity chromatography and the purified protein is injected, intraperitoneally, into BALB/c mice to induce antibody production. It may be advantageous to add an adjuvant, such as Freund's adjuvant, to increase the resulting immune response.

Serum is tested for the production of specific antibodies and spleen cells from animals having a positive specific antibody titer are used for cell fusions with myeloma cells to generate hybridoma clones. Supernatants derived from hybridoma clones are tested for the presence of monoclonal antibodies having specificity against NET-4. For a general description of monoclonal antibody methodology, see, e.g., Harlow and Lane, Antibodies: A Laboratory Manual, Cold Spring Harbor Laboratory (1988).

In addition to the baculovirus expression system, other suitable bacterial or yeast expression systems may be employed for the expression of NET-4 protein or polypeptides thereof. As with the baculovirus system, it may be advantageous to utilize one of the commercially available affinity tags to facilitate purification prior to inoculation of the animals. Thus, the NET-4 cDNA or fragment thereof may be isolated by, e.g., agarose gel purification and ligated in frame with a suitable tag protein such as 6-His, glutathione-S-transferase (GST) or other such readily available affinity tag. See, e.g., Molecular Biotechnology: Principles and Applications of Recombinant DNA, ASM Press pp. 160-161 (ed. Glick, B. R. and Pasternak, J. J. 1998).

In other embodiments of the present invention, NET-4 modulators are humanized anti-NET-4 monoclonal antibodies. The phrase “humanized antibody” refers to an antibody derived from a non-human antibody—typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci., U.S.A., 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which patents are incorporated herein by reference.

It will be appreciated that alternative NET-4 inhibitor antibodies may be readily obtained by other methods commonly known in the art. One exemplary methodology for identifying antibodies having a high specificity for NET-4 is the phage display technology.

Phage display libraries for the production of high-affinity antibodies are described in, for example, Hoogenboom, H. R. et al., Immunotechnology 4(1):1-20 (1998); Hoogenboom, H. R., Trends Biotechnol. 15:62-70 (1997) and McGuinness, B. et al., Nature Bio. Technol. 14:1149-1154 (1996) each of which is incorporated herein by reference. Among the advantages of the phage display technology is the ability to isolate antibodies of human origin that cannot otherwise be easily isolated by conventional hybridoma technology. Furthermore, phage display antibodies may be isolated in vitro without relying on an animal's immune system.

Antibody phage display libraries may be accomplished, for example, by the method of McCafferty et al., Nature 348:552-554 (1990) which is incorporated herein by reference. In short, the coding sequence of the antibody variable region is fused to the amino terminus of a phage minor coat protein (pIII). Expression of the antibody variable region-pIII fusion construct results in the antibody's “display” on the phage surface with the corresponding genetic material encompassed within the phage particle.

NET-4 protein suitable for screening a phage library may be obtained by, for example, expression in baculovirus Sf9 cells as described, supra. Alternatively, the NET-4 coding region may be PCR amplified using primers specific to the desired region of the NET-4 protein. A desired region can be selected based on the structure shown in FIG. 1. As discussed above, the NET-4 protein may be expressed in E. coli or yeast as a fusion with one of the commercially available affinity tags.

The resulting fusion protein may then be adsorbed to a solid matrix, e.g., a tissue culture plate or bead. Phage expressing antibodies having the desired anti-NET-4 binding properties may subsequently be isolated by successive panning, in the case of a solid matrix, or by affinity adsorption to a NET-4 antigen column. Phage having the desired NET-4 inhibitory activities may be reintroduced into bacteria by infection and propagated by standard methods known to those skilled in the art. See Hoogenboom, H. R., Trends Biotechnol., supra for a review of methods for screening for positive antibody-pill phage.

Small Molecules

The present invention also provides small molecule NET-4 modulators (antagonists and agonists) that may be readily identified through routine application of high-throughput screening (HTS) methodologies. Persidis, A., Nature Biotechnology 16:488-489 (1998). HTS methods generally refer to those technologies that permit the rapid assaying of lead compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Lead compounds may be identified via incorporation of radioactivity or through optical assays that rely on absorbance, fluorescence or luminescence as read-outs. Gonzalez, J. E. et al., Curr. Opin. Biotechnol. 9(6):624-631 (1998) incorporated herein by reference.

HTS methodology may be employed, e.g., to screen for lead compounds that block one of NET-4's biological activities, particularly its ability to interact with other cell-surface proteins. By this method, NET-4 protein may be immunoprecipitated from cells expressing the protein and applied to wells on an assay plate suitable for robotic screening. Individual test compounds may then be contacted with the immunoprecipitated protein and the effect of each test compound on NET-4 activity assessed.

Methods for Assessing the Efficacy of NET-4 Modulators

Lead molecules or compounds, whether antisense molecules or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to inhibit NET-4 gene expression or biological activity. As discussed in further detail in the Examples, NET-4 inhibitors of the present invention are effective in reducing NET-4 expression levels and inhibiting cancer cell proliferation. Thus, the present invention further discloses methods that permit the skilled artisan to assess the effect of candidate inhibitors on each of these parameters.

As noted above and as presented in the Examples, candidate NET-4 inhibitors may be tested by administration to cells that either express endogenous NET-4 or that are made to express NET-4 by transfection of a mammalian cell, such as SW620, with a recombinant NET-4 plasmid construct.

Effective NET-4 inhibitory molecules will reduce the levels of NET-4 mRNA as determined, e.g., by Northern blot or RT-PCR analysis. Example 1; Sambrook et al., Molecular Cloning: A Laboratory Manual Cold Spring Harbor Press (1989) and Molecular Biotechnology: Principles and Applications of Recombinant DNA, ASM Press (ed. Glick, B. R. and Pasternak, J. J. 1998) incorporated herein by reference, or may reduce the levels of NET-4 protein in the cell. The effectiveness of a given candidate antisense molecule may be assessed by comparison with a control “antisense” molecule known to have no substantial effect on NET-4 expression when administered to a mammalian cell. Exemplary control molecules include the RC oligonucleotides disclosed in Example 2.

NET-4 inhibitors effective in reducing NET-4 gene expression and/or cell proliferation by one or more of the methods discussed herein may be further characterized in vivo for efficacy in one of the readily available animal model systems. The various animal model systems for study of cancer and genetic instability associated genes are discussed in, for example, Donehower, L. A. Cancer Surveys 29:329-352 (1997) incorporated herein by reference.

Administration of NET-4 Inhibitors and Compositions Thereof

The present invention provides NET-4 inhibitors and compositions comprising one or more NET-4 inhibitor as well as methods that employ these inventive inhibitors in in vivo, ex vivo, and in vitro applications where it is advantageous to reduce or eliminate the expression or activity of NET-4 or a functionally downstream molecule. NET-4 inhibitors may find use as drugs for supplementing cancer therapeutics and other agents. NET-4 inhibitors may also find use in other diseases of hyperproliferation.

Compositions may be administered parenterally, topically, orally or locally for therapeutic treatment. Preferably, the compositions are administered orally or parenterally, i.e., intravenously, intraperitoneally, intradermally or intramuscularly.

Inventive compositions will include one or more NET-4 inhibitor and may further comprise a pharmaceutically acceptable carrier or excipient. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

NET-4 inhibitors useful in the treatment of disease in mammals will often be prepared substantially free of other naturally occurring immunoglobulins or other biological molecules. Preferred NET-4 inhibitors will also exhibit minimal toxicity when administered to a mammal.

The compositions of the invention may be sterilized by conventional, well known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride and stabilizers (e.g., 1-20% maltose, etc.).

The selection of the appropriate method for administering NET-4 inhibitors of the present invention will depend on the nature of the application envisioned as well as the nature of the NET-4 inhibitor. Thus, for example, the precise methodology for administering a NET-4 inhibitor will depend upon whether it is an antisense molecule, a protein and/or peptide, an antibody or antibody fragment or a small molecule. Other considerations include, for example, whether the NET-4 inhibitor will be used to inhibit tumor cell growth, invasion, or metastasis, or as an adjunct to other cancer therapeutics.

A variety of methods are available in the art for the administration of antisense molecules. Exemplary methods include gene delivery techniques, including both viral and non-viral based methods as well as liposome mediated delivery methods.

Gene delivery methodologies will be effective to, for example, reduce tumor cell proliferation, or supplement radiation and/or chemotherapeutic treatment of tumors. Wheldon, T. E. et al., Radiother Oncol 48(1):5-13 (1998) (gene delivery methodologies for enhancement of fractionated radiotherapy). By these methodologies, substantial therapeutic benefit may be achieved despite transfection efficiencies significantly less than 100%, transient retention of the transfected inhibitor and/or existence of a subpopulation of target cells refractory to therapy.

Alternatively, gene delivery methodology may be used to directly knock out endogenous NET-4 within tumor cells. For example, the NET-4 gene may be targeted by transfection of a gene delivery vector carrying a NET-4 inhibitor. Preferential transfection into or expression within tumor cells may be achieved through use of a tissue-specific or cell cycle-specific promoter, such as, e.g., promoters for prostate-specific antigen or for immunoglobulin genes (Vile, R. G. et al., Cancer Res. 53:962-967 (1993) and Vile, R. G., Semin. Cancer Biol. 5:437-443 (1994)) or through the use of trophic viruses that are confined to particular organs or structures, such as, e.g., a replication selective and neurotrophic virus that can only infect proliferating cells in the central nervous system.

Thus, to achieve therapeutic benefit, NET-4 within the tumor cells should be preferentially inhibited. This can be accomplished by transfecting a gene expressing a NET-4 inhibitor, a NET-4 antisense molecule, a NET-4 gene specific repressor, or an inhibitor of the protein product of the NET-4 gene.

As used herein, the phrase “gene delivery vector” refers generally to a nucleic acid construct that carries and, within certain embodiments, is capable of directing the expression of an antisense molecule of interest, as described in, for example, Molecular Biotechnology: Principles and Applications of Recombinant DNA, Ch. 21, pp. 555-590 (ed. B. P. Glick and J. J. Pasternak, 2^{nd} ed. 1998); Jolly, Cancer Gene Ther. 1:51-64 (1994); Kimura, Human Gene Ther. 5:845-852 (1994); Connelly, Human Gene Ther. 6:185-193 (1995); and Kaplitt, Nat. Gen. 6:148-153 (1994).

A number of virus and non-virus based gene delivery vector systems have been described that are suitable for the administration of NET-4 inhibitors. Virus based gene delivery systems include, but are not limited to retrovirus, such as Moloney murine leukemia virus, spumaviruses and lentiviruses; adenovirus; adeno-associated virus; and herpes-simplex virus vector systems. Viruses of each type are readily available from depositories or collections such as the American Type Culture Collection (ATCC; 10801 University Boulevard, Manassas, Va. 20110-2209) or may be isolated from known sources using commonly available materials and techniques.

The gene delivery vector systems of the present invention will find applications both in in vivo as well as ex vivo therapeutic regimens. Each of these applications is described in further detail below.

1. Retroviral Gene Delivery Vector Systems

Within one aspect of the present invention, retroviral gene delivery vectors are provided that are constructed to carry or express a NET-4 inhibitory antisense molecule. As used herein, the term “NET-4 inhibitory antisense molecule” refers generally to a nucleic acid sequence having NET-4 inhibitory activity. More specifically, such antisense molecules will reduce NET-4 gene expression. Retroviral gene delivery vectors of the present invention may be readily constructed from a wide variety of retroviruses, including for example, B, C, and D type retroviruses as well as spumaviruses and lentiviruses. See RNA Tumor Viruses, Cold Spring Harbor Laboratory (2^{nd} ed. 1985).

Any of the above retroviruses may be readily utilized in order to assemble or construct retroviral gene delivery vectors given the disclosure provided herein, and standard recombinant DNA techniques. See, e.g., Sambrook et al, Molecular Cloning: A Laboratory Manual, Cold Spring Harbor Laboratory Press (2d ed. 1989) and Kunkle, Proc. Natl. Acad. Sci. U.S.A. 82:488 (1985). In addition, within certain embodiments of the invention, portions of the retroviral gene delivery vectors may be derived from different retroviruses.

A retroviral vector, suitable for the expression of a NET-4 inhibitory antisense molecule, must include at least one transcriptional promoter/enhancer or locus defining element(s), or other elements that control gene expression by other means such as alternate splicing, nuclear RNA export, post-translational modification of messenger, or post-transcriptional modification of protein. Such vector constructs must also include a packaging signal, long terminal repeats (LTRs) or portion thereof, and positive and negative strand primer binding sites appropriate to the retrovirus used (if these are not already present in the retroviral vector). Optionally, the retroviral vector may also include a signal that directs polyadenylation, selectable markers such as Neomycin resistance, TK, hygromycin resistance, phleomycin resistance histidinol resistance, or DHFR, as well as one or more restriction sites and a translation termination sequence. Within one aspect of the present invention, retroviral gene delivery vector constructs are provided comprising a 5′ LTR, a tRNA binding site, a packaging signal, one or more heterologous sequences, an origin of second strand DNA synthesis and a 3′ LTR, wherein the vector construct lacks gag/pol or env coding sequences.

Other retroviral gene delivery vectors may likewise be utilized within the context of the present invention, including, for example, those disclosed in the following each of which is incorporated herein by reference: EP 0,415,731; WO 90/07936; WO 94/03622; WO 93/25698; WO 93/25234; U.S. Pat. No. 5,219,740; WO 93/11230; WO 93/10218; Vile et al., Cancer Res. 53:3860-3864 (1993); Vile et al., Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127, GB 2,200,651, EP 0,345,242 and WO 91/02805.

Packaging cell lines suitable for use with the above-described retroviral gene delivery vector constructs may be readily prepared. See, e.g., U.S. Pat. Nos. 5,716,832 and 5,591,624. These packaging cell lines may be utilized to create producer cell lines (also termed vector cell lines or “VCLs”) for the production of recombinant vector particles. It may be preferred to use packaging cell lines made from human (e.g., HT1080 cells) or mink parent cell lines, thereby allowing production of recombinant retroviruses that avoid inactivation in human serum.

2. Adeno-Associated Viral Gene Delivery Vector Systems

Adeno-associated viruses (AAV) possess a number of qualities that make them particularly suitable for the development of gene delivery vectors generally and for the delivery of polynucleotides encoding NET-4 inhibitory antisense molecules in particular. For a general review of AAV expression systems, see Rabinowitz et al., Current Opin. Biotech. 9(5):470-475 (1998). AAV is a non-pathogenic, defective human parvovirus that is non-infective without an adeno or herpes helper virus. Thus, in the absence of a helper virus, AAV becomes integrated latently into the host genome. In addition, AAV has the advantage over the retroviruses, discussed above, in being able to transduce a wide range of both dividing and quiescent cell types.

A variety of AAV gene delivery vectors may be utilized to direct the expression of one or more NET-4 inhibitor antisense molecule. Representative examples of such vectors include the AAV vectors disclosed by Srivastava in WO 93/09239; Samulski, et al. J. Virol. 63:3822-3828 (1989); Mendelson, et al. Virol. 166:154-165 (1988); and Flotte, et al. Proc. Natl. Acad. Sci. U.S.A. 90(22):10613-10617 (1993) incorporated herein by reference.

Briefly, an AAV gene delivery vector of the present invention may include, in order, a 5′ adeno-associated virus inverted terminal repeat; a polynucleotide encoding the NET-4 inhibitory antisense molecule; a sequence operably linked to the NET-4 inhibitory antisense molecule that regulates its expression in a target tissue, organ or cell; and a 3′ adeno-associated virus inverted terminal repeat. A suitable regulatory sequence for the expression of NET-4 inhibitory antisense molecule is, e.g., the enhancer/promoter sequence of cytomegalovirus (CMV). In addition, the AAV vector may preferably have a polyadenylation sequence such as the bovine growth hormone (BGH) polyadenylation sequence.

Generally, AAV vectors should have one copy of the AAV ITR at each end of the NET-4 inhibitory antisense molecule, to allow replication, packaging, efficient integration into the host cell genome and rescue from the chromosome. The 5′ ITR sequence consists of nucleotides 1 to 145 at the 5′ end of the AAV DNA genome, and the 3′ ITR includes nucleotides 4681 to 4536 of the AAV genome. Preferably, the AAV vector may also include at least 10 nucleotides following the end of the ITR (i.e., a portion of the so-called “D region”).

Optimal packaging of an adeno-associated virus gene delivery vector requires that the 5′ and 3′ ITRs be separated by approximately 2-5 kb. It will be apparent, however, that the ideal spacing between ITR sequences may vary depending on the particular packaging system utilized. This spacing may be achieved by incorporating a “stuffer” or “filler” polynucleotide fragment to bring the total size of the nucleic acid sequence between the two ITRs to between 2 and 5 kb. Thus, where the NET-4 inhibitory antisense molecule is smaller than 2-5 kb, a non-coding stuffer polynucleotide may be incorporated, for example, 3′ to the 5′ ITR sequence and 5′ of the NET-4 inhibitory antisense molecule. The precise nucleotide sequence of the stuffer fragment is not an essential element of the final construct.

Depending upon the precise application contemplated, rather than incorporating a stuffer fragment, multiple copies of the NET-4 inhibitory antisense molecule may be inserted, inter alia, to achieve the optimal ITR sequence spacing. It may be preferred to organize the polynucleotides as two or more separate transcription units each with its own promoter and polyadenylation signal.

Recombinant AAV vectors of the present invention may be generated from a variety of adeno-associated viruses, including for example, serotypes 1 through 6. For example, ITRs from any AAV serotype are expected to have similar structures and functions with regard to replication, integration, excision and transcriptional mechanisms.

Within certain embodiments of the invention, expression of the NET-4 inhibitory antisense molecule may be accomplished by a separate promoter (e.g., a viral promoter). Representative examples of suitable promoters in this regard include a CMV promoter, an RSV promoter, an SV40 promoter, or a MoMLV promoter. Other promoters that may similarly be utilized within the context of the present invention include cell or tissue specific promoters or inducible promoters. Representative inducible promoters include tetracycline-response promoters (e.g., the “Tet” promoter) as described in Gossen et al., Proc. Natl. Acad. Sci. U.S.A. 89:5547-5551 (1992); Gossen et al., Science 268:1766-1769 (1995); Baron et al., Nucl. Acids Res. 25:2723-2729 (1997); Blau et al., Proc. Natl. Acad. Sci. U.S.A. 96:797-799 (1999); Bohl et al., Blood 92:1512-1517 (1998); and Haberman et al., Gene Therapy 5:1604-1611 (1998); the ecdysone promoter system as described in No et al., Proc. Natl. Acad. Sci. U.S.A. 93:3346-3351 (1996); and other regulated promoters or promoter systems as described in Rivera et al., Nat. Med. 2:1028-1032 (1996).

The AAV gene delivery vector may also contain additional sequences, for example from an adenovirus, which assist in effecting a desired function for the vector. Such sequences include, for example, those which assist in packaging the AAV gene delivery vector in adenovirus particles.

Packaging cell lines suitable for producing adeno-associated viral vectors may be routinely prepared given readily available techniques. See, e.g., U.S. Pat. No. 5,872,005, incorporated herein by reference. At a minimum, suitable packaging systems for AAV gene delivery systems of the present invention will include the AAV replication and capsid genes.

Preferred packaging cell lines may contain both an AAV helper virus as well as an AAV gene delivery vector containing the NET-4 inhibitory antisense molecule. For detailed descriptions of representative packaging cell line systems, see, e.g. Holscher, C. et al., J. Virol. 68:7169-7177 (1994); Clark, K. R. et al., Hum. Gene Ther. 6:1329-1341 (1995); and Tamayosa, K. et al., Hum. Gen. Ther. 7:507-513 (1996) which are incorporated herein by reference.

Alternatively, packaging of AAV may be achieved in vitro in a cell free system to obviate transfection protocols or packaging cell lines. Such in vitro systems incorporate an AAV gene delivery vector bearing the NET-4 inhibitory antisense molecule and a source of Rep-protein, capsid-protein and Adenovirus proteins that supply helper-viral functions. The latter proteins are typically supplied in the form of a cell extract. Representative in vitro systems are further described in Ding, L. et al., Gen. Ther. 4:1167-1172 (1997) and Zhou, Z. et al., J. Virol. 72:3241-3247 (1998) which are incorporated herein by reference.

3. Other Viral Gene Delivery Vector Systems

In addition to retroviral vectors and adeno-associated virus-based vectors, numerous other viral gene delivery vector systems may also be utilized for the expression of NET-4 inhibitory antisense molecules. For example, within one embodiment of the invention adenoviral vectors may be employed. Representative examples of such vectors include those described by, for example, Berkner, Biotechniques 6:616-627 (1988); Rosenfeld et al., Science 252:431-434 (1991); WO 93/9191; Kolls et al., Proc. Natl. Acad. Sci. U.S.A. 91(1):215-219 (1994); Kass-Eisler et al., Proc. Natl. Acad. Sci. U.S.A. 90(24):11498-502 (1993); Guzman et al., Circulation 88(6):2838-48 (1993); Guzman et al., Cir. Res. 73(6):1202-1207 (1993); Zabner et al., Cell 75(2):207-216 (1993); Li et al., Hum. Gene Ther. 4(4):403-409 (1993); Caillaud et al., Eur. J. Neurosci. 5(10):1287-1291 (1993); Vincent et al., Nat. Genet. 5(2):130-134 (1993); Jaffe et al., Nat. Genet. 1(5):372-378 (1992); and Levrero et al., Gene 101(2):195-202 (1991); and WO 93/07283; WO 93/06223; and WO 93/07282.

Gene delivery vectors of the present invention also include herpes vectors. Representative examples of such vectors include those disclosed by Kit in Adv. Exp. Med. Biol. 215:219-236 (1989); and those disclosed in U.S. Pat. No. 5,288,641 and EP 0176170 (Roizman). Additional exemplary herpes simplex virus vectors include HFEM/ICP6-LacZ disclosed in WO 95/04139 (Wistar Institute), pHSVlac described in Geller, Science 241:1667-1669 (1988), and in WO 90/09441 and WO 92/07945; HSV Us3::pgC-lacZ described in Fink, Human Gene Therapy 3:11-19 (1992); and HSV 7134, 2 RH 105 and GAL4 described in EP 0453242 (Breakefield), and those deposited with the ATCC as accession numbers ATCC VR-977 and ATCC VR-260.

Gene delivery vectors may also be generated from a wide variety of other viruses including, for example, poliovirus (Evans et al., Nature 339:385-388 (1989); and Sabin, J. Biol. Standardization 1:115-118 (1973)); rhinovirus; pox viruses, such as canary pox virus or vaccinia virus (Fisher-Hoch et al., Proc. Natl. Acad. Sci. U.S.A. 86:317-321 (1989); Flexner et al., Ann. N.Y. Acad. Sci. 569:86-103 (1989); Flexner et al., Vaccine 8:17-21 (1990); U.S. Pat. Nos. 4,603,112, 4,769,330 and 5,017,487; WO 89/01973); SV40 (Mulligan et al., Nature 277:108-114 (1979); influenza virus (Luytjes et al., Cell 59:1107-1113 (1989); McMicheal et al., N. Eng. J. Med. 309:13-17 (1983); and Yap et al., Nature 273:238-239 (1978)); HIV (Poznansky, J. Virol. 65:532-536 (1991)); measles (EP 0 440,219); astrovirus (Munroe et al., J. Vir. 67:3611-3614 (1993)); and coronavirus, as well as other viral systems (e.g., EP 0,440,219; WO 92/06693; U.S. Pat. No. 5,166,057).

4. Non-viral Gene Delivery Vectors

Other gene delivery vectors and methods that may be employed for the expression of NET-4 inhibitory antisense molecules such as, for example, nucleic acid expression vectors; polycationic condensed DNA linked or unlinked to killed adenovirus alone, for example, see Curiel, Hum Gene Ther 3:147-154 (1992); ligand linked DNA, for example, see Wu, J Biol Chem 264:16985-16987 (1989); eucaryotic cell delivery vectors; deposition of photopolymerized hydrogel materials; hand-held gene delivery particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol Cell Biol 14:2411-2418 (1994), and in Woffendin, Proc. Natl. Acad. Sci. 91:1581-1585 (1994).

Particle mediated gene delivery may be employed. Briefly, the NET-4 inhibitory antisense molecule of interest can be inserted into conventional vectors that contain conventional control sequences for high level expression, and then be incubated with synthetic gene delivery molecules such as polymeric DNA-binding cations like polylysine, protamine, and albumin, linked to cell targeting ligands such as asialoorosomucoid, as described in Wu, et al., J. Biol. Chem. 262:4429-4432 (1987), insulin as described in Hucked, Biochem Pharmacol 40:253-263 (1990), galactose as described in Plank, Bioconjugate Chem 3:533-539 (1992), lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, PCT Patent Publication Nos. WO 95/13796, WO 94/23697, and WO 91/144445, and European Patent Publication No. 524,968. Nucleic acid sequences can be inserted into conventional vectors that contain conventional control sequences for high level expression, and then be incubated with synthetic gene delivery molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al., Proc. Natl. Acad. Sci. U.S.A. 91(24):11581-11585 (1994). Moreover, the coding sequence and the product of expression of such can be delivered through deposition of photopolymerized hydrogel materials.

Exemplary liposome and polycationic gene delivery vehicles are those described in U.S. Pat. Nos. 5,422,120 and 4,762,915, in PCT Patent Publication Nos. WO 95/13796, WO 94/23697, and WO 91/14445, in European Patent Publication No. 524,968 and in Starrier, Biochemistry, pp. 236-240 (1975) W. H. Freeman, San Francisco; Shokai, Biochem. Biophys. Acta. 600:1 (1980); Bayer, Biochem. Biophys. Acta. 550:464 (1979); Rivet, Methods Enzymol. 149:119 (1987); Wang, Proc. Natl. Acad. Sci. U.S.A. 84:7851 (1987); Plant, Anal. Biochem. 176:420 (1989).

The polynucleotides of the invention can be formulated as a diagnostic kit for detecting, for example, the expression of NET-4 messenger RNA in a tumor cell. A diagnostic kit may contain at least one oligonucleotide capable of hybridizing to SEQ ID NO:1 under stringent conditions. Preferably the polynucleotide will be at least 10 base pairs in length. In a preferred embodiment, the kit will comprise at least one oligonucleotide selected from the group consisting of SEQ ID Nos:18, 19, 20, 21 and 22, and at least one control oligonucleotide that does not hybridize with a polynucleotide of SEQ ID NO:1 under stringent conditions. The kit may also comprise at least 3 of these oligonucleotides, such as all five oligonucleotides.

### EXAMPLES

The following experimental examples are offered by way of illustration, not limitation.

### Example 1

### Antisense Inhibition of NET-4 mRNA

A. Preparation of Transfection Mixture

For each transfection mixture, a carrier molecule, preferably a lipitoid or cholesteroid, was prepared to a working concentration of 0.5 mM in water, sonicated to yield a uniform solution, and filtered through a 0.45 μm PVDF membrane. The antisense oligonucleotide (SEQ ID NO:2-6) was prepared to a working concentration of 100 μM in sterile Millipore water.

The oligonucleotide was diluted in OptiMEM™ (Gibco/BRL), in a microfuge tube, to 2 μM, or approximately 20 μg oligo/ml of OptiMEM™. In a separate microfuge tube, lipitoid or cholesteroid, typically in the amount of about 1.5-2 nmol lipitoid/μg antisense oligonucleotide, was diluted into the same volume of OptiMEM™ used to dilute the oligonucleotide. The diluted antisense oligonucleotide was immediately added to the diluted lipitoid and mixed by pipetting up and down.

B. Transfection

SW620 or MRC9 cells were plated on tissue culture dishes one day in advance of transfection, in growth media with serum, to yield a density at transfection of 60-90%. The oligonucleotide/lipitoid mixture was added to the cells, immediately after mixing, to a final concentration of 100-300 nM antisense oligonucleotide. Cells were incubated with the transfection mixture at 37° C., 5% CO₂ for 4-24 hours. After incubation, the transfection mixture was removed and replaced with normal growth media with serum.

Total RNA was extracted using the RNEASY™ kit (Qiagen Corporation, Chatsworth, Calif.), according to manufacturer's protocols.

C. Reverse Transcription

The level of target mRNA was quantitated using the Roche LightCycler™ real-time PCR machine. Values for the target mRNA were normalized versus an internal control (e.g., beta-actin).

For each 20 μl reaction, extracted RNA (generally 0.2-1 μg total) was placed into a sterile 0.5 or 1.5 ml microcentrifuge tube, and water was added to a total volume of 12.5 μl. To each tube was added 7.5 μl of a buffer/enzyme mixture, prepared by mixing (in the order listed) 2.5 μl H₂O, 2.0 μl 10× reaction buffer, 10 μl oligo dT (20 pmol), 1.0 μl dNTP mix (10 mM each), 0.5 μl RNAsin® (20 u) (Ambion, Inc., Hialeah, Fla.), and 0.5 μl MMLV reverse transcriptase (50 u) (Ambion, Inc.). The contents were mixed by pipetting up and down, and the reaction mixture was incubated at 42° C. for 1 hour. The contents of each tube were centrifuged prior to amplification.

D. LightCycler™ Amplification of RT Reactions

An amplification mixture was prepared by mixing in the following order: 1× PCR buffer II, 3 mM MgCl₂, 140 μM each dNTP, 0.175 pmol each oligo, 1:50,000 dil of SYBR® Green, 0.25 mg/ml BSA, 1 unit Taq polymerase, and H₂O to 20 μl. (PCR buffer II is available in 10× concentration from Perkin-Elmer, Norwalk, Conn.). In 1× concentration it contains 10 mM Tris pH 8.3 and 50 mM KCl. SYBR® Green (Molecular Probes, Eugene, Ore.) is a dye which fluoresces when bound to double stranded DNA. As double stranded PCR product is produced during amplification, the fluorescence from SYBR® Green increases.

To each 20 μl aliquot of amplification mixture, 2 μl of template RT was added, and amplification was carried out according to standard protocols.

As shown in Table 1 below, NET-4 message levels were decreased relative to actin message in SW620 cells. AS represents the antisense oligonucleotides and RC represents the corresponding control oligonucleotides.

TABLE 1
Effect of
NET-4 Oligonucleotides on NET-4 mRNA Levels in SW620 Cells

| Antisense | NET-4 message levels |
|---|---|
| oligonucleotide | normalized to actin |
| SEQ ID NO:2 AS | 0.04 |
| SEQ ID NO:3 AS | 0.06 |
| SEQ ID NO:4 AS | 0.07 |
| SEQ ID NO:5 AS | 0.03 |
| SEQ ID NO:6 AS | 0.02 |
| SEQ ID NO:7 RC | 0.27 |
| SEQ ID NO:8 RC | 0.32 |
| SEQ ID NO:9 RC | 0.35 |
| SEQ ID NO:10 RC | 0.54 |
| SEQ ID NO:11 RC | 0.26 |

### Example 2

### Cell Proliferation Assay

Cells were seeded into 96 well plates at a density of 5000 cells per well. For a 4 day proliferation assay, 5 independent 96 well plates were prepared, one for each day. After overnight incubation, cells were transfected using the procedure described above. On each day of the proliferation assay, all medium was removed from one plate and frozen at −70° C. On day four, all plates were developed with the Quantos™ assay kit (Stratagene, La Jolla, Calif.) which determines the amount of DNA, and thus the number of cells, in each well. The results are shown in Tables 2-6 below. In each table, the results are expressed in units of fluorescence.

TABLE 2
Effect of NET-4 Oligonucleotide 509 on Growth of SW620 Cells

| Oligonucleotide | Day 0 | Day 1 | Day 2 | Day 3 | Day 4 |
|---|---|---|---|---|---|
| Wild type (no oligo) | 1100 | 2300 | 2600 | 3800 | 4400 |
| 509AS (SEQ ID NO:2) | 1100 | 1500 | 1300 | 1600 | 2500 |
| 509RC (SEQ ID NO:7) | 1300 | 1300 | 1700 | 2500 | 2900 |

TABLE 3
Effect of NET-4 Oligonucleotide 353 on Growth of SW620 Cells

| Oligonucleotide | Day 0 | Day 1 | Day 2 | Day 3 | Day 4 |
|---|---|---|---|---|---|
| Wild type (no oligo) | 1100 | 2300 | 2700 | 3800 | 4300 |
| 353AS (SEQ ID NO:3) | 1000 | 1500 | 1000 | 1400 | 2500 |
| 353RC (SEQ ID NO:8) | 1300 | 1700 | 1900 | 2300 | 2300 |

TABLE 4
Effect of NET-4 Oligonucleotide 446 on Growth of SW620 Cells

| Oligonucleotide | Day 0 | Day 1 | Day 2 | Day 3 | Day 4 |
|---|---|---|---|---|---|
| Wild type (no oligo) | 1100 | 2400 | 2700 | 3800 | 4400 |
| 446AS (SEQ ID NO:4) | 1000 | 1700 | 1300 | 1800 | 2800 |
| 446RC (SEQ ID NO:9) | 1300 | 2400 | 2600 | 3500 | 4000 |

TABLE 5
Effect of NET-4 Oligonucleotide 960 on Growth of SW620 Cells

| Oligonucleotide | Day 0 | Day 1 | Day 2 | Day 3 | Day 4 |
|---|---|---|---|---|---|
| Wild type (no oligo) | 1000 | 2300 | 2700 | 3800 | 4400 |
| 960AS (SEQ ID NO:5) | 1000 | 1100 | 1000 | 1100 | 1700 |
| 960RC (SEQ ID NO:11) | 1200 | 1700 | 2000 | 2600 | 3400 |

TABLE 6
Effect of NET-4 Oligonucleotide 960 on Growth of MRC9 Cells

| Oligonucleotide | Day 0 | Day 1 | Day 2 | Day 3 | Day 4 |
|---|---|---|---|---|---|
| Wild type (no oligo) | 400 | 1000 | 1200 | 1700 | 2100 |
| 960AS (SEQ ID NO:5) | 450 | 800 | 800 | 1100 | 1090 |
| 960RC (SEQ ID NO:11) | 400 | 600 | 800 | 1250 | 1300 |

TABLE 7
Effect of NET-4 Oligonucleotide 999 on Growth of SW620 Cells

| Oligonucleotide | Day 0 | Day 1 | Day 2 | Day 3 | Day 4 |
|---|---|---|---|---|---|
| Wild type (no oligo) | 1100 | 2400 | 2700 | 3800 | 4400 |
| 999AS (SEQ ID NO:6) | 1100 | 1700 | 1300 | 1700 | 2600 |
| 999RC (SEQ ID No:11) | 1300 | 2000 | 2000 | 2900 | 3100 |

The oligonucleotides are:

| | | |
|---|---|---|
| hNET-4: 509AS | CTGGGTCAAAGCCGCCGAGATCG | (SEQ ID NO:2) |
| hNET-4: 509RC | GCTAGAGCCGCCGAAACTGGGTC | (SEQ ID NO:7) |
| hNET-4: 353AS | TTCAGGACCCTTGTAGTGCTTCCCG | (SEQ ID NO:3) |
| hNET-4: 353RC | GCCCTTCGTGATGTTCCCAGGACTT | (SEQ ID NO:8) |
| hNET-4: 446AS | CCATGCCCACAGTCCAATTCCAAGA | (SEQ ID NO:4) |
| hNET-4: 446RC | AGAACCTTAACCTGACACCCGTACC | (SEQ ID NO:9) |
| hNET-4: 960AS | GCTGGTCAACTTCTGGTTTTTGCCT | (SEQ ID NO:5) |
| hNET-4: 960RC | TCCGTTTTTGGTCTTCAACTGGTCG | (SEQ ID NO:10) |
| hNET-4: 999AS | AAACTGGGGCACACAGCCTTTCGT | (SEQ ID NO:6) |
| hNET-4: 999RC | TGCTTTCCGACACACGGGGTCAAA | (SEQ ID NO:11) |

### Example 3

### NET-4 mRNA Expression

Quantitative PCR of a number of normal tissues and tumor cell lines was used to analyze expression of Net-4. Quantitative real-time PCR was performed by first isolating RNA from cells using a Roche RNA Isolation kit according to manufacturer's directions. One microgram of RNA was used to synthesize a first-strand cDNA using MMLV reverse transcriptase (Ambion) using the manufacturer's buffer and recommended concentrations of oligo dT, nucleotides, and Rnasin. This first-strand cDNA served as a template for quantitative real-time PCR using the Roche light-cycler as recommended in the machine manual. Net-4 was amplified with the forward primer GATCCCGCAGAAGATGTCATCAACA (SEQ ID NO:12) and the reverse primer TCTGCAGCAATGCAATGCCTATGA (SEQ ID NO:13). PCR product was quantified based on the cycle at which the amplification entered the linear phase of amplification in comparison to an internal standard and using the software supplied by the manufacturer. Small differences in amounts or total template in the first-strand cDNA reaction were eliminated by normalizing to amount of actin amplified in a separate quantitative PCR reaction using the forward primer 5′-CGGGAAATCGTGCGTGACATTAAG-3′ (SEQ ID NO:14) and the reverse primer: 5′-TGATCTCCTTCTGCATCCTGTCGG-3′ (SEQ ID NO:15). The results for Net-4 mRNA levels in normal tissues are shown in Table 8; the results for Net-4 mRNA levels in tumor cell lines are shown in Table 9.

TABLE 8
Net-4 mRNA Levels in Normal Tissues

| | Net-4 mRNA Levels normalized |
|---|---|
| Tissue type | to glucuronidase B mRNA levels |
| Brain(whole) | 9.0 |
| Heart | 1.3 |
| Kidney | 3.3 |
| Liver | 0.2 |
| Lung | 0.6 |
| Colon | 0.4 |
| Bone marrow | 4.2 |
| Small intestine | 0.6 |
| Spleen | 0.2 |
| Stomach | 0.3 |
| Thymus | 1 |
| Prostate | 0.8 |
| Skeletal muscle | 1.6 |
| Testis | 1.6 |
| Uterus | 4.6 |
| Fetal brain | 14.1 |
| Fetal liver | 8.2 |
| Spinal cord | 6.8 |
| Placenta | 0.4 |
| Adrenal gland | 0.5 |
| Pancreas | 0.4 |
| Salivary gland | 2.5 |
| Trachea | 1.4 |
| Mammary gland | 1.5 |

TABLE 9
Net-4 mRNA Levels in Tumor Cell Lines

| | | Net-4 mRNA |
|---|---|---|
| | | levels normalized |
| | | to actin |
| Cell line | Description of cell line | mRNA levels |
| MDA-MB- | Human breast cancer (metastatic to | 0.80 |
| 231 | bone and/or lung) | |
| MDA-MB- | Human breast cancer (metastatic to | 0.07 |
| 435 | lung) | |
| Alab | Human breast cancer (highly | 0.02 |
| | metastatic) | |
| MCF-7 | Human breast cancer (metastatic to | 0.34 |
| | bone and/or lung) | |
| MDA-MB- | Human breast adenocarcinoma | 1.16 |
| 468 | (metastatic) | |
| 184B5 | Normal breast epithelial cells, | 0.18 |
| | chemically transformed | |
| HMVEC | Human microvascular endothelial cells | 1.64 |
| DU 145 | Human prostate cancer (metastatic to | 1.32 |
| | brain) | |
| HT1080 | Human fibrosarcoma | 1.55 |
| SKOV3 | Human ovarian cancer | 0.79 |
| OVCAR3 | Human ovarian cancer | 0.07 |
| Km12C | Human colon cancer (lowly metastatic) | 0.21 |
| Km12L4 | Human colon cancer (highly metastatic) | 0.43 |
| 293 | Human kidney transformed with | 0.86 |
| | adenovirus 5 DNA | |
| Caco.2 | Human intestinal epithelial cell | 0.36 |
| SW620 | Human colon adenocarcinoma | 1.18 |
| | (metastatic to lymph node) | |
| LS174T | Human colon cancer | 2.00 |
| LOVO | Human colon adenocarcinoma | 0.61 |
| | (metastatic to left supraclavicular | |
| | region) | |
| HT29 | Human colon adenocarcinoma | 1.09 |
| SW480 | Human colon adenocarcinoma | 1.29 |
| | (metastatic to lymph node) | |
| HCT116 | Human colon cancer | 7.11 |
| Colo 320 | Human colon cancer | 4.30 |
| DN | | |
| T84 | Human colon cancer (metastatic to | 0.57 |
| | lung) | |
| HCT15 | Human colorectal cancer | 0.93 |
| DLD-1 | Human colon adenocarcinoma | 0.75 |
| LNCAP | Human prostatic cancer (metastic to left | 0.64 |
| | supraclavicular lymph node) | |
| WOCA | Primary prostate epithelium | 1.21 |
| PC3 | Human prostate cancer | 2.46 |
| GRDP2 | Primary prostate epithelium | 2.79 |
| U373MG | Human glioblastoma | 0.46 |
| IMR90 | Human lung fibroblasts | 1.75 |

### Example 4

### Antisense Inhibition of Cell Growth

The effect of Net-4 expression upon colony formation was tested in a soft agar assay. Soft agar assays were conducted by first establishing a bottom layer of 2 ml of 0.6% agar in media plated fresh within a few hours of layering on the cells. The cell layer was formed on the bottom layer by removing cells transfected as described above from plates using 0.05% trypsin and washing twice in media. The cells were counted in a Coulter counter, and resuspended to 10⁶ per ml in media. 10 μl aliquots were placed with media in 96-well plates (to check counting with WST1), or diluted further for soft agar assay. 2000 cells were plated in 800 μl 0.4% agar in duplicate wells above 0.6% agar bottom layer. After the cell layer agar solidified, 2 ml of media was dribbled on top and antisense or reverse control oligo was added without delivery vehicles. Fresh media and oligos were added every 3-4 days. Colonies formed in 10 days to 3 weeks. Fields of colonies were counted by eye. Wst-1 metabolism values were used to compensate for small differences in starting cell number.

As shown in Table 10, antisense oligonucleotides to Net-4 (960AS; SEQ ID NO:5) led to decreased colony number compared to reverse control oligonucleotides (960RC; SEQ ID NO:10). The decrease in colony number by antisense oligonucleotide 960AS (SEQ ID NO:5) is similar to that by antisense oligonucleotide kRAS 2576AS (GCATGTGGAAGGTAGGGAGGCAAGA (SEQ ID NO:16), which is directed to the kRAS mRNA, when compared to control reverse oligonucleotides kRAS 2576RC (AGAACGGAGGGATGGAAGGTGTACG (SEQ ID NO:17).

TABLE 10
Effect of Net-4 Oligonucleotides on the Growth of SW620 cells

| | Growth of SW 620 Cells |
|---|---|
| | (number of colonies |
| | normalized to starting cell |
| | numbers as measured by |
| Oligonucleotide | WTS1 metabolism) |
| 960AS (SEQ ID NO:5) | 245 |
| 960RC (SEQ NO:10) | 470 |
| kRAS 2576AS (SEQ ID NO:16) | 270 |
| kRAS 2576RC (SEQ ID NO:17) | 400 |

### Example 5

### Colon Microarray Analysis

Normal and cancerous tissues were collected from patients using laser capture microdissection (LCM) techniques, which are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001).

cDNA probes were prepared from total RNA isolated from the patient cells. Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling. Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red).

Each array used had an identical spatial layout and control spot set. Each microarray was divided into two areas, each area having an array with, on each half, twelve groupings of 32×12 spots for a total of about 9,216 spots on each array. The two areas are spotted identically which provide for at least two duplicates of each clone per array. Spotting was accomplished using PCR amplified products from 0.5 kb to 2.0 kb and spotted using a Molecular Dynamics Gen III spotter according to the manufacturer's recommendations. The first row of each of the 24 regions on the array had about 32 control spots, including 4 negative control spots and 8 test polynucleotides. The test polynucleotides were spiked into each sample before the labeling reaction with a range of concentrations from 2-600 pg/slide and ratios of 1:1. For each array design, two slides were hybridized with the test samples reverse-labeled in the labeling reaction. This provided for about 4 duplicate measurements for each clone, two of one color and two of the other, for each sample.

The differential expression assay was performed by mixing equal amounts of probes from tumor cells and normal cells of the same patient. The arrays were prehybridized by incubation for about 2 hrs at 60° C. in 5× SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5× SSC, and 0.2% SDS. After hybridization, the array was washed at 55° C. three times as follows: 1) first wash in 1× SSC/0.2% SDS; 2) second wash in 0.1× SSC/0.2% SDS; and 3) third wash in 0.1× SSC.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to normal. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level of fluorescence for each sequence on the array was expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. During initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.” Database tables were populated using a 95% confidence level (p>0.05).

The difference in the expression level of Net-4 in the colon tumor cells relative to the matched normal colon cells was greater than or equal to 2 fold (“>=2×”) in 91.67% of the patients, greater than or equal to 2.5 fold in 66.67% of the patients, and greater than or equal to 5 fold in 33.33% of the patients examined.

### Example 6

### In Situ Hybridization Analysis

In situ hybridization analysis was conducted to characterize the expression of Net-4 in normal colon and lung tissues, as well as tumor colon and lung tissues using a mixture of the following oligonucleotides:

| | | |
|---|---|---|
| NET-4 oligo 864 | CCCTTGTAGTGC | (SEQ ID NO:18) |
| NET-4 oligo 865 | TGAAGTATTTGATGC | (SEQ ID NO:19) |
| NET-4 oligo 866 | CCGATATGCTCTG | (SEQ ID NO:20) |
| NET-4 oligo 867 | TTAGGTTCCAATCA | (SEQ ID NO:21) |
| NET-4 oligo 868 | CGACTTTCATACTG | (SEQ ID NO:22) |

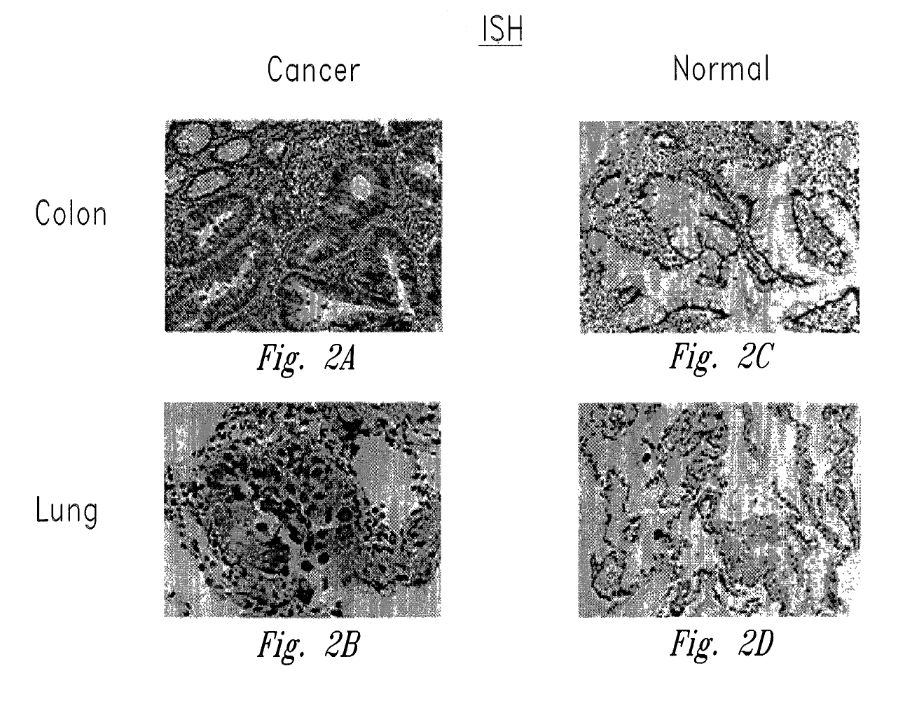

The results are shown in FIG. 2 (FIG. 2A—tumor colon tissue, FIG. 2B—tumor lung tissue, FIG. 2C—normal colon tissue, and FIG. 2D—normal lung tissue).

From the foregoing it will be appreciated that, although specific embodiments of the invention have been described herein for purposes of illustration, various modifications may be made without departing from the spirit and scope of the invention. Accordingly, the invention is not limited except as by the appended claims.

What is claimed is:

1. An isolated NET-4 modulator selected from the group consisting of an antisense oligonucleotide, a ribozyme, a protein, a polypeptide, and a small molecule.

2. The isolated NET-4 modulator of claim 1 wherein said NET-4 modulator is an antisense molecule.

3. The isolated NET-4 modulator of claim 2 wherein said antisense molecule or the complement thereof comprises at least 15 consecutive nucleic acids of the sequence of SEQ ID NO:1.

4. The isolated NET-4 modulator of claim 3 wherein said antisense molecule or the complement thereof hybridizes under high stringency conditions to the sequence of SEQ ID NO:1.

5. The isolated NET-4 modulator of claim 2 wherein said antisense molecule comprises a nucleic acid sequence selected from the group consisting of SEQ ID NO:2-6.

6. The isolated NET-4 modulator of claim 1 wherein said NET-4 modulator is selected from the group consisting of an antibody and an antibody fragment.

7. The isolated NET-4 modulator of claim 6 wherein said antibody or antibody fragment is monoclonal.

8. The isolated NET-4 modulator of claim 7 wherein said antibody or antibody fragment is humanized.

9. A composition, comprising a therapeutically effective amount of a NET-4 modulator in a pharmaceutically acceptable carrier.

10. The composition of claim 9, comprising two or more NET-4 modulators.

11. The composition of claim 9 wherein said NET-4 modulator is an antisense molecule.

12. A method of decreasing the expression of NET-4 in a mammalian cell, comprising administering to said cell a NET-4 inhibitor of claim 1.

13. The method of claim 12 wherein said NET-4 modulator is an antisense molecule.

14. The method of claim 13 wherein said antisense molecule or the complement thereof comprises at least 15 consecutive nucleic acids of the sequence of SEQ ID NO:1.

15. The method of claim 13 wherein said antisense molecule or the complement thereof hybridizes under high stringency conditions to the sequence of SEQ ID NO:1.

16. The method of claim 13 wherein said antisense molecule comprises a nucleic acid sequence selected from the group consisting of SEQ ID NO:2-6.

17. The method of claim 12 wherein said NET-4 modulator is selected from the group consisting of a protein and a polypeptide.

18. The method of claim 12 wherein said NET-4 modulator is a small molecule.

19. The method of claim 12 wherein said NET-4 modulator is administered ex vivo to said mammalian cell.

20. A method of treating neoplastic disease, comprising administering to a mammalian cell a NET-4 modulator of claim 1 such that said neoplastic disease is reduced in severity.

21. A diagnostic kit for detecting the presence of a colon tumor cell in a biological sample, said kit comprising at least one oligonucleotide selected from the group consisting of SEQ ID NOs:18, 19, 20, 21 and 22, and at least one control oligonucleotide that does not hybridize with a polynucleotide of SEQ ID NO:1 under stringent conditions.

22. The kit of claim 21 comprising at least 3 of said oligonucleotides.

23. The kit of claim 21 comprising said five oligonucleotides.
